(19) United States
(12) Patent Application Publication
(10) Pub. No.: US 2023/0165782 A1
(43) Pub. Date: Jun. 1, 2023

(54) COMPOSITION FOR IMPROVING DAMAGED HAIR

(71) Applicant: BEAUTEE COLLAGEN CO., LTD., Cheongju-si, Chungcheongbuk-do (KR)

(72) Inventor: Kwan Mo LEE, Cheongju-si (KR)

(73) Assignee: BEAUTEE COLLAGEN CO., LTD., Cheongju-si, Chungcheongbuk-do (KR)

(21) Appl. No.: 17/921,783

(22) PCT Filed: Apr. 28, 2020
(86) PCT No.: PCT/KR2020/005596

§ 371 Date: Oct. 27, 2022

(51) Int. Cl. A61K 8/88; A61Q 5/00; A61K 8/65

(52) U.S. Cl. CPC A61K 8/88; A61Q 5/002; A61K 8/65; A61K 2800/594

(57) ABSTRACT

The present invention relates to a composition which includes a polymer compound and a neutralizing agent and can be applied to damaged hair, wherein the compound included in the composition has unshared electron pairs and thus forms strong polar bonds with alpha-hydrogen in amino acids of the hair to which the compound is to be bonded, the composition has both a hard segment and a soft segment in the compound, and thus imparts multifunctionality, such as elasticity, repellency, and bulkiness, and collagen included in the composition attaches to damaged cell membrane complexes (CMC), thus enabling the damaged hair to recover. Further, oxygen included in the compound forms hydrogen bonds with moisture in the air to form a film having high antistatic properties and a large refractive index, and thus imparts gloss and deep-color effects.

### CROSS-REFERENCE TO RELATED APPLICATIONS

This application is a national phase under 35 U.S.C. § 371 of PCT International Application No. PCT/KR2020/005596 which has an International filing date of Apr. 28, 2020, the entire contents of each of which are hereby incorporated by reference.

### TECHNICAL FIELD

The present invention relates to a composition that includes a polymer compound, collagen, and a neutralizing agent, and can be applied to damaged hair.

### BACKGROUND ART

The tip of the hair is exposed to the external environment longer than the hair around the scalp, so the tip of the hair is damaged more. Weathering caused by such exposure to the external environment results in loss of the hair cuticle, thereby causing split ends and eventually leading to hair breakage. The case in which hair tension decreases and becomes weak when high-temperature heat is continuously applied to the hair through a hair dryer and the like is an example.

Hair damage caused by various physical, chemical, or environmental factors due to frequent perms and dyeing, causes proteins and lipids in the hair to be eluted, resulting in hair with high porosity. As a result, hair becomes rough and lusterless, and friction force increases, thereby making hair grooming difficult. In addition, hair is easily broken or split at the end of the hair. Furthermore, there is also a problem of a decrease in hair volume or hair thinning due to drooping hair caused by loss of elasticity.

In order to solve such problems, various products for improving damaged hair, such as hair treatments, are used. However, such products contain substances harmful to the human body, including the scalp, apart from the effect of hair protection, which is a problem. For example, in the case of hair products containing silicone, there may be an environmental problem since microplastics can be detected. Hence, research on new materials has been requested for the improvement of damaged hair, but the reality of the situation is that the research is still insufficient.

### DOCUMENT OF RELATED ART

### Patent Document

Korean Patent Application Publication No. 10-2012-0037922

### DISCLOSURE

### Technical Problem

In order to solve the above problems, the inventors of the present invention have studied a composition that is harmless to the environment and to the human body while improving the condition of damaged hair. As a result, the invention has been completed with the use of a neutralizing agent and a multifunctional polymer compound having both a soft segment and a hard segment. In addition, the present invention can be applied not only to hair but also to animal hair, such as wool, cashmere, and goat hair, and wigs.

Hence, the present invention is to provide a composition for improving damaged hair, the composition including a polymer compound including a polymerization unit represented by Formula 1, a neutralizing agent, and collagen.

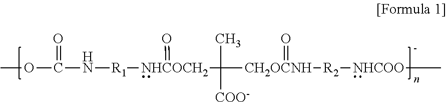

(In Formula 1, R₁ and R₂ are each independently a saturated or unsaturated straight-chain or branched-chain alkyl group having 1 to 2 carbon atoms, and n is an integer in a range of 1 to 5.)

### Technical Solution

In order to achieve an objective of the present invention, the present invention provides a composition for improving damaged hair, the composition including a polymer compound including a polymerization unit represented by Formula 1, a neutralizing agent, and collagen.

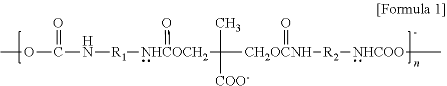

(In Formula 1, R₁ and R₂ are each independently a saturated or unsaturated straight-chain or branched-chain alkyl group having 1 to 2 carbon atoms, and n is an integer in a range of 1 to 5.)

According to an embodiment of the present invention, the neutralizing agent may be an amine salt.

According to an embodiment of the present invention, the amine salt may be represented by Formula 2.

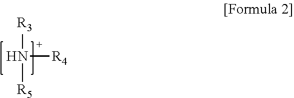

(in Formula 2, R₃ to R₅ are each independently a saturated or unsaturated straight-chain or branched-chain alkyl group having 1 to 5 carbon atoms.)

According to an embodiment of the present invention, the polymer compound, the neutralizing agent, and the collagen included in the composition may account for 15% to 40% by weight with respect to a total weight of the composition.

According to an embodiment of the present invention, a weight ratio of the polymer compound, the neutralizing agent, and the collagen of the composition may be in a range of 1:0.05 to 0.1:0.5 to 1.5.

According to an embodiment of the present invention, the composition may not contain microplastics.

### Advantageous Effects

A compound included in a composition of the present invention has unshared electron pairs and thus forms strong polar bonds with alpha-hydrogen of amino acids in hair to which the compound is to be bonded, and has both a hard segment and a soft segment and thus imparts multifunctionality, such as elasticity, repellency, and bulkiness. In addition, collagen included in the composition attaches to damaged cell membrane complexes (CMCs) and thus repairs damaged hair, and oxygen included in the compound forms hydrogen bonds with moisture in the air to form a film having good antistatic properties and a high refractive index and thus imparts gloss and deep-color effects to hair. Furthermore, since there are no microplastics, the composition is eco-friendly and harmless to the human body. Moreover, the composition can be applied not only to hair but also to animal hair, such as wool, cashmere, and goat hair, and wigs.

### DESCRIPTION OF DRAWINGS

FIG. 1 shows observation results of a composition of the present invention and a hair product in the market, the observation performed with a scanning transmission electron microscope after applying the composition and the hair product on a wafer and drying the same;

FIG. 2 shows IR analysis results for confirmation of a structure of a compound of the present invention; and

FIGS. 3 to 9 confirm the effect of a composition of the present invention on the improvement of damaged hair.

### BEST MODE

The inventors of the present invention have studied a composition that is harmless to the human body while improving damage of damaged hair and consequently, have completed the present invention by applying a multifunctional polymer compound that has both a soft segment and a hard segment to the present invention.

Hence, the present invention provides a composition for improving damaged hair, the composition including a polymer compound including a polymerization unit represented by Formula 1, a neutralizing agent, and collagen.

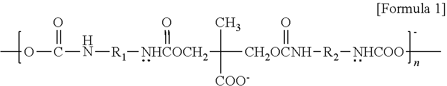

(In Formula 1, R₁ and R₂ are each independently a saturated or unsaturated straight-chain or branched-chain alkyl group having 1 to 2 carbon atoms, and n is an integer in a range of 1 to 5.)

In Formula 1, R₁ and R₂ may be each independently a straight-chain saturated alkyl group having one carbon atom.

In the present invention, the compound represented by Formula 1 has both the hard segment and soft segment and thus imparts multifunctionality, such as elasticity, repellency, and bulkiness. In addition, the collagen included in the composition attaches to damaged cell membrane complexes (CMCs) and thus enables damaged hair to be repaired. Furthermore, oxygen included in the compound forms hydrogen bonds with moisture in the air to form a film having good antistatic properties and a high refractive index and thus imparts gloss and deep-color effects to hair.

In the present invention, the concept of the hair is applied not only to human hair but also to artificial hair, and also includes animal hair, such as wool, cashmere, and the like.

In Formula 1, R₁ and R₂ are each independently the saturated or unsaturated straight-chain or branched-chain alkyl group having 1 to 2 carbon atoms, and n is the number of a repeating unit. The average number of the repeating unit is in a range of 1 to 5, but is not limited thereto.

In the present invention, the neutralizing agent may be an amine salt. Preferably, the neutralizing agent is represented by Formula 2, but is not limited thereto.

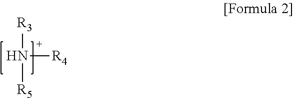

(In Formula 2, R₃ to R₅ are each independently a saturated or unsaturated straight-chain or branched-chain alkyl group having 1 to 5 carbon atoms.)

In Formula 2, R₃ to R₅ may be each independently a straight-chain saturated alkyl group having three carbon atoms. In addition, in Formula 2, R₃ to R₅ may be each independently an alkyl group in which one or more hydrogen are replaced with —OH.

In the present invention, improving damaged hair means at least reducing hair damage, for example, thinning hair, split ends, loss of gloss and softness, or the like.

In the present invention, the collagen is a fibrous protein that can be easily found in most animals, particularly, mammals, and constitutes all of the connective tissues in the body, such as skin, cartilage, and the like. Collagen forms a rope-like structure in which three polypeptide molecules are twisted into a triple helix. In each polypeptide chain, glycine and proline are alternately arranged with each other, and several different amino acids are inserted randomly in between. Typically, there are hydroxylysine and hydroxyproline derived from lysine and proline, respectively. These two play an important role in the formation of a quaternary structure in which chains are tightly connected to each other through hydrogen bonds.

In the present invention, collagen is not particularly limited, and preferably, includes fish collagen.

The collagen is attached to the cell membrane complexes (CMCs) of the hair to repair damaged hair.

In the present invention, the polymer compound, the neutralizing agent, and the collagen included in the composition may account for 15% to 40% by weight with respect to a total weight of the composition.

In the present invention, a weight ratio of the polymer compound, the neutralizing agent, and the collagen in the composition may be in a range of 1:0.05 to 0.1:0.5 to 1.5. Preferably, the weight ratio thereof is 1:0.075:0.75. When the weight ratio of the polymer compound, the neutralizing agent, and the collagen in the composition is the above weight ratio, the effect of improving damaged hair according to the present invention is maximized.

In the present invention, the composition may not contain microplastics. The microplastics refer to synthetic polymer compounds having a size of 5 mm or less which are purposefully manufactured or miniaturized from existing products by being fragmented into pieces. Such microplastics not only negatively affect the environment but also absorb external pollutants, which can negatively affect the human body when remaining on the skin.

The composition of the present invention can be manufactured in many different forms in order to prevent hair from being damaged and to provide gloss and softness to hair. The composition can be applied to all cosmetic compositions for hair, for example, shampoos, conditioners, hair treatments, hair colorants, bleaching agents, hair gels, hair conditioners, hair tonics, hair creams, hair waxes, hair sprays, hair mousses, permanent agents, hair serums, massage creams, and the like. In addition, the composition can be applied to sprays or spray-type products.

Furthermore, the composition of the present invention may further include typical optional components, which are well known to those skilled in the art, for example, moisturizers, fatty substances, wetting agents, oils, fragrances, preservatives, vitamins, proteins, and the like, in addition to the above components in order to maintain basic physical properties and qualities depending on the formulation.

### MODE FOR INVENTION

Hereinafter, the present invention will be described in further detail with reference to Examples and Comparative Examples, which is only for the purpose of explanation of the present invention and does not limit the scope of the present invention.

### Example 1: Preparation of Compound and Collagen of Present Invention

Preparation of Compound of Present Invention

0.5 L of 1,4-butanediol, 200 g of trimethylolpropane (TMP), 100 g of isophorone diisocyanate, and 300 g of polyester polyol (having an MW of 2000) were mixed and then stirred at a temperature of 50° C. for 30 minutes. Next, 300 g of polyethylene Glycol (having an MW of 1000), 1 L of cyclohexanedimethanol (CHDM), and 2,2-bis(hydroxymethyl)propanoic acid (DMPA) were additionally treated and mixed at a temperature of 40° C. for 30 minutes with a stirrer to complete the preparation of a compound of the present invention.

Preparation of Collagen of Present Invention

Collagen raw material (pollack skin) was cut into small pieces and hydrolyzed with water (in a weight ratio of the pollack skin to the water of 1:90) by using pepsin (in an amount of 1% by weight and 50,000 units). Then, the sludge was removed by a separator, and 30% of a non-volatile phase was made by filtration and concentration. By performing hydrolysis in a high-pressure mechanical device at a pressure of 700 bar for an hour and performing spray drying with the addition of 0.5% pepsin, the preparation of a collagen powder was completed.

### Example 2: IR Analysis of Compound of Present Invention

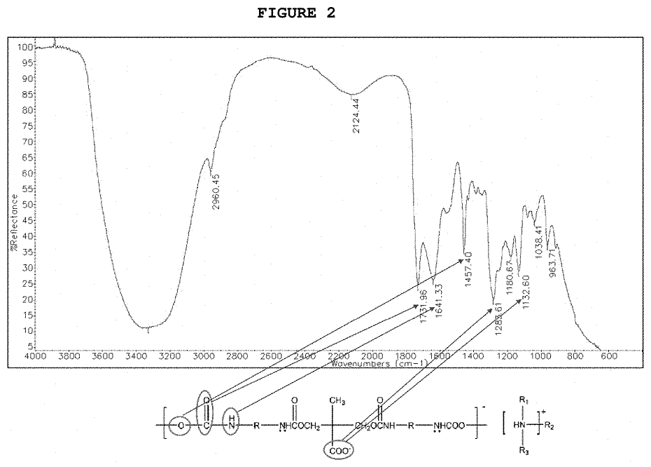

In order to confirm a structure of the above-prepared compound, IR analysis was performed with a device (SP-300 purchased from OPTIMA) according to a method described in a user guide. As a result, as shown in FIG. 2, it was confirmed that the main functional groups in the compound of the present invention existed.

### Example 3: Preparation of Composition of Present Invention

A composition for improving damaged hair of the present invention was prepared by using the compound obtained from Example 1, which is represented by Formula 1, and collagen. Specifically, 20% by weight of the compound of the present invention, 1.5% by weight of triethanolamine, 15% by weight of the collagen, 41.5% by weight of distilled water, 20% by weight of glycerine, and 2% by weight of a preservative were stirred at a temperature of 30° C. for an hour and dissolved.

### Example 4: Examination of Presence and Absence of Microplastics in Composition of Present Invention

The above-prepared composition and a hair product in the market were diluted 10-fold, evenly applied on an analysis wafer of a scanning transmission electron microscope (EVO MA 10 purchase from Carl Zeiss), and naturally dried for 24 hours. Then, analysis was performed with the above microscope at 700 times and 500 times magnification.

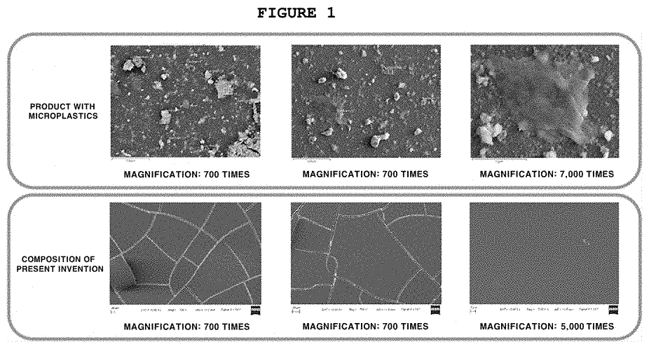

As a result, as shown in FIG. 1, in the case of the composition of the present invention, it was confirmed that no microplastics were found in both magnifications. On the other hand, in the case of the hair product in the market, it was confirmed that microplastics were found.

### <Example 5: Confirmation of Effect of Composition of Present Invention on Improvement of Damaged Hair

With the composition prepared in Example 3, the effect of the composition of the present invention on improvement of damaged hair was confirmed.

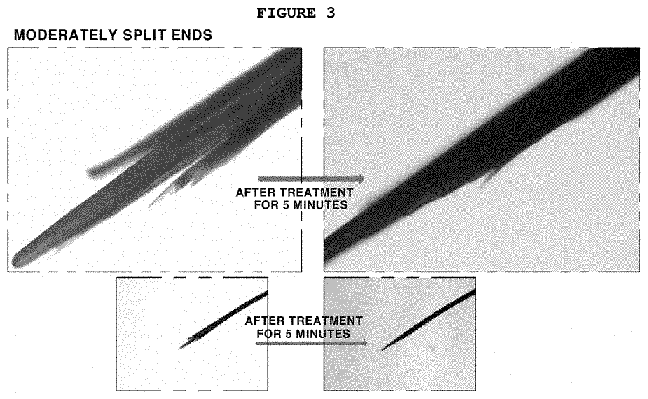

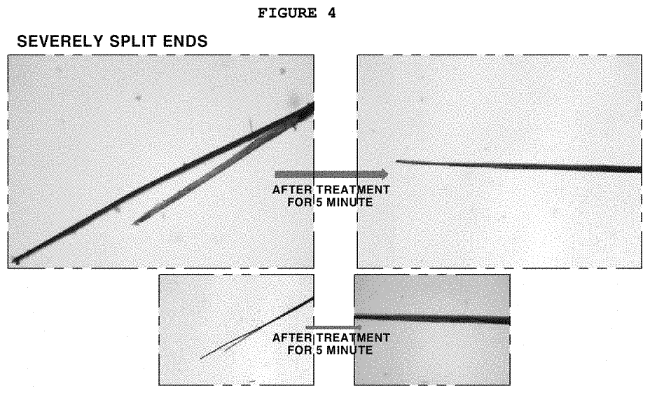

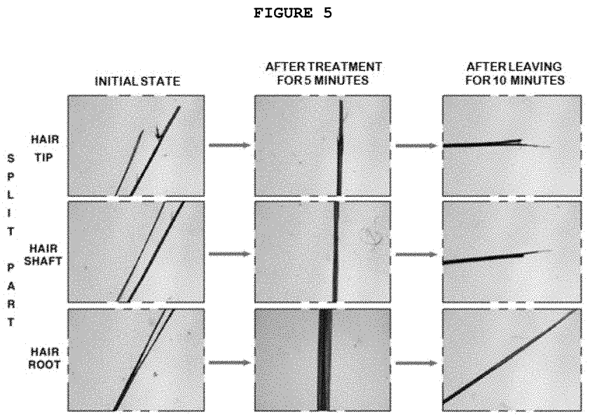

First, the composition was diluted to 10%, then treated on damaged hair for 5 minutes to 10 minutes, and analyzed at high magnification and low magnification. As a result, as shown in FIGS. 3 to 5, it was confirmed that split ends of hair were improved.

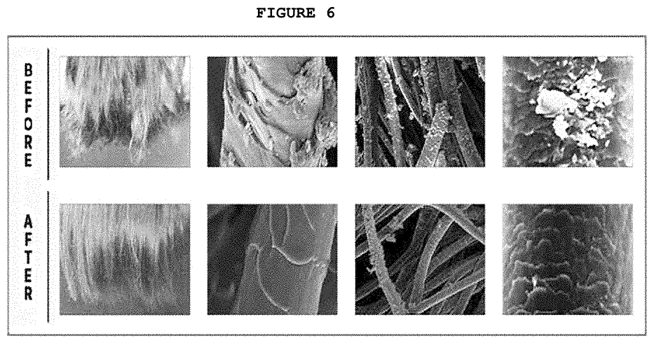

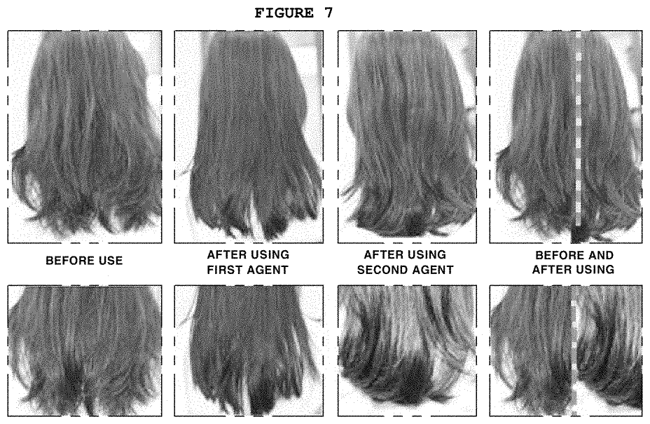

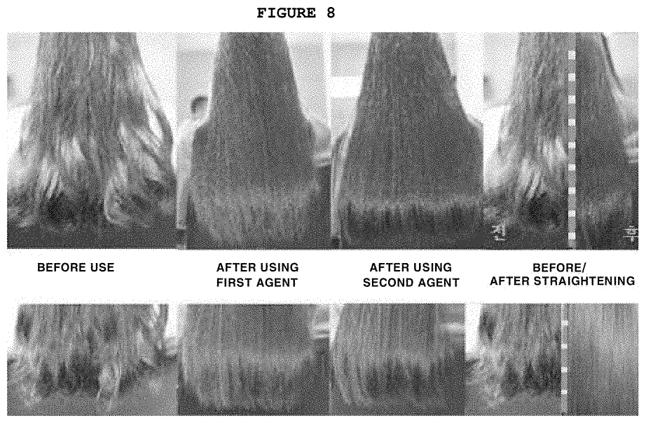

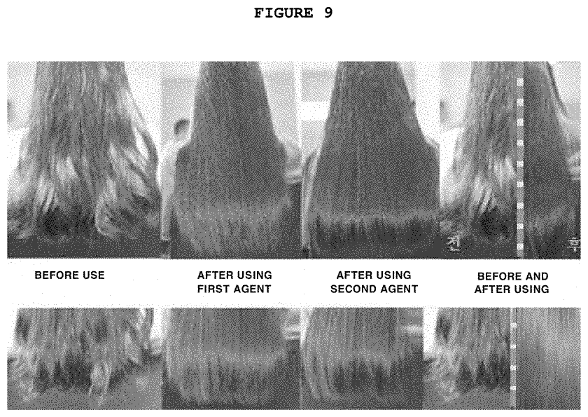

In addition, the composition diluted to 3% was treated on damaged hair to confirm whether the damage to the hair after being chemically processed was prevented or not. As a result, as shown in FIGS. 6 to 9, when the composition of the present invention was treated, it was confirmed that the damage to the hair after being chemically processed was reduced and gloss, bulkiness, and deep-color effects were excellent.

### INDUSTRIAL APPLICABILITY

A compound included in a composition of the present invention has unshared electron pairs and thus forms strong polar bonds with alpha-hydrogen of amino acids in hair to which the compound is to be bonded, and has both a hard segment and a soft segment and thus imparts multifunctionality, such as elasticity, repellency, and bulkiness. In addition, collagen included in the composition attaches to damaged cell membrane complexes (CMCs) and thus repairs damaged hair, and oxygen included in the compound forms hydrogen bonds with moisture in the air to form a film having good antistatic properties and a high refractive index and thus imparts gloss and deep-color effects to hair. Furthermore, since there are no microplastics, the composition is eco-friendly and harmless to the human body. Moreover, the composition can be applied not only to hair but also to animal hair, such as wool, cashmere, and goat hair, and wigs.

1. A composition for improving damaged hair, the composition comprising a polymer compound comprising a polymerization unit represented by Formula 1, a neutralizing agent, and collagen. 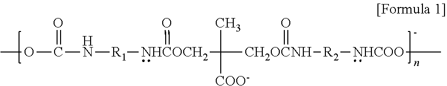
(In Formula 1, R₁ and R₂ are each independently a saturated or unsaturated straight-chain or branched-chain alkyl group having 1 to 2 carbon atoms, and n is an integer in a range of 1 to 5)

2. The composition of claim 1, wherein the neutralizing agent is an amine salt.

3. The composition of claim 2, wherein the amine salt is represented by Formula 2. 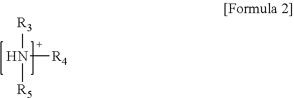
(In Formula 2, R₃ to R₅ are each independently a saturated or unsaturated straight-chain or branched-chain alkyl group having 1 to 5 carbon atoms.)

4. The composition of claim 1, wherein the polymer compound, the neutralizing agent, and the collagen contained in the composition accounts for 15% to 40% by weight with respect to a total weight of the composition.

5. The composition of claim 1, wherein a weight ratio of the polymer compound, the neutralizing agent, and the collagen in the composition is in a range of 1:0.05 to 0.1:0.5 to 1.5.

6. The composition of claim 1, wherein the composition does not contain microplastics.
